(19) United States
(12) Patent Application Publication
(10) Pub. No.: US 2010/0317033 A1
(43) Pub. Date: Dec. 16, 2010

(54) METHODS AND MATERIALS FOR DETECTING FOOD CONTAINING ALLERGENS

(76) Inventor: Matthew Philip Abdel, Rochester, MN (US)

Correspondence Address: FISH & RICHARDSON P.C. (TC), PO BOX 1022, MINNEAPOLIS, MN, 55440-1022, US

(21) Appl. No.: 12/813,182

(22) Filed: Jun. 10, 2010

Related U.S. Application Data
(60) Provisional application No. 61/185,691, filed on Jun. 10, 2009.

(51) Int. Cl. G01N 33/53

(52) U.S. Cl. 435/7.9; 435/7.1

(57) ABSTRACT

This document provides methods and materials related to detecting allergens (e.g., food allergens) and/or specific antibodies in individualized consumers that are specific to an allergen (e.g., a food allergen). For example, methods and materials for assessing a food sample for the presence or absence of food allergens are provided. In addition, methods and materials for assessing if a particular human will react with those exact food allergens given his/her antibody profile are provided.

### CROSS-REFERENCE TO RELATED APPLICATIONS

This application claims the benefit of priority to U.S. Provisional Application Ser. No. 61/185,691, filed Jun. 10, 2009. The disclosure of the prior application is considered part of (and is incorporated by reference in) the disclosure of this application.

### BACKGROUND

1. Technical Field

This document relates to methods and materials involved in detecting allergens (e.g., food allergens) in products and methods and materials involved in detecting antibodies in humans that may make them susceptible to allergic reactions. For example, this document provides methods and materials for assessing a food sample for the presence or absence of food allergens and methods and materials for evaluating whether or not a particular allergen will interact with a specific individual.

2. Background Information

Many people suffer from allergies to a particular type of food or component that can be found in food. For example, many people are allergic to peanuts or peanut components. The severity of a food allergy can range from fairly mild discomfort to serious life threatening responses (e.g., anaphylaxis).

### SUMMARY

This document relates to methods and materials involved in detecting allergens (e.g., food allergens) in products and methods and materials involved in detecting antibodies in humans that may make them susceptible to allergic reactions. For example, this document provides methods and materials for assessing a food sample for the presence or absence of food allergens and methods and materials for evaluating whether or not a particular allergen will interact with a specific individual.

As described herein, a personal allergy detector device can be configured to provide a user with the ability to assess a product such as food (e.g., food at the point of purchase or consumption) for the presence or absence of one or more allergens (e.g., food allergens) quickly and accurately. Such a device can allow a user to consume the tested food with an increased confidence that the food is free of the particular food allergens of that user. In some cases, a personal allergy detector device provided herein can be a self contained device that includes one or more sample inlet ports such that all test reagents and inserted samples remain within the device. Such self contained devices can be configured for single use and can be disposable.

In some cases, a personal allergy detector device provided herein can be configured to assess antibodies in a human (e.g., a consumer) that may react with allergens in food, thus leading to an allergic reaction. This assessment can be completed quickly and accurately. Such a device can allow a user to consume the tested food with an increased confidence that he/she will not react with that particular food.

In general, one aspect of this document features a method for testing food for the presence of a food allergen using a solid support having a first zone, a second zone, and a third zone. The method comprises, or consists essentially of, (a) contacting the first zone of the solid support with a food sample, wherein the first zone comprises multiple different anti-food allergen antibodies, wherein each different food allergen antibody has binding specificity for a different food allergen and each different food allergen antibody comprises an enzyme, wherein the contacting is performed under conditions wherein the food allergen, if present, and the anti-food allergen antibody having binding specificity for the food antigen form a complex, (b) advancing material from the first zone to the second zone, wherein the second zone comprises multiple immobilized different anti-food allergen antibodies and a substrate for the enzyme, wherein the complex, if present in the material, binds to the immobilized anti-food allergen antibody having binding specificity for the food allergen of the complex, and wherein the enzyme of the complex interacts with the substrate to form a detectable label in the second zone, wherein the presence of the detectable label in the second zone indicates that the food contains the food allergen, and (c) advancing material from the second zone to the third zone, wherein the third zone comprises immobilized anti-antibody antibodies having binding specificity for the anti-food allergen antibodies and a substrate for the enzyme, wherein the anti-food allergen antibodies present in the material binds to the immobilized anti-antibody antibodies, and wherein the enzyme of the anti-food allergen antibody bound to immobilized anti-antibody antibody interacts with the substrate to form a detectable label in the third zone, wherein the presence of the detectable label in the third zone indicates that test was completed. The food allergen can be a peanut, tree nut, walnut, almond, pecan, pistachio, hazelnut, Brazil nut, Macadamia nut, cashew, beechnut, chestnut, hickory nut, filberts, soy, milk, egg, wheat, or shellfish. The method can be performed in a restaurant setting. The enzyme can be horseradish peroxidase.

In another aspect, this document features a method for testing food for the presence of a food allergen specific to a human. The method comprises, or consists essentially of, (a) contacting a solid support comprising an immobilized anti-human IgE antibody with a food sample to be tested and a saliva sample from the human under conditions wherein the food allergen if present within the food sample binds to a human IgE antibody present in the saliva if the human is allergic to the food allergen and under conditions wherein a human IgE antibody if present within the saliva binds to the immobilized anti-human IgE antibody, thereby forming a complex comprising the anti-human IgE antibody, the human IgE antibody, and the food allergen if the human IgE antibody is present in the saliva and the food allergen is present in the food sample, (b) contacting the solid support with an anti-food allergen antibody comprising a detectable label under conditions wherein the anti-food allergen antibody binds to the complex if present, (c) removing unbound forms of the anti-food allergen antibody, and (d) detecting the detectable label, wherein the presence of the detectable label indicates that the food sample contains the food allergen. The food allergen can be a peanut, tree nut, walnut, almond, pecan, pistachio, hazelnut, Brazil nut, Macadamia nut, cashew, beechnut, chestnut, hickory nut, filberts, soy, milk, egg, wheat, or shellfish. The method can be performed in a restaurant setting. The anti-human IgE antibody can be a monoclonal antibody. The anti-food allergen antibody can be a polyclonal antibody. The step (b) can comprise contacting the solid support with multiple different anti-food allergen antibodies, wherein each of the multiple different anti-food allergen antibody comprises a detectable label and has a binding specificity for a different food allergen, and wherein each of the anti-food allergen antibody binds to the complex if the complex is present and if the complex comprises the food allergen for which the anti-food allergen antibody has binding specificity. The step (c) can comprise washing the solid support to remove the unbound forms of the anti-food allergen antibody. The solid support can be configured to have a reservoir housing a wash solution. The detectable label can be propidium iodide, fluorescein, fluorescein isothiocyanate, tetramethylrhodamine isothiocyanate, rhodamine, R-phycoerythrin, or aminomethylcoumarin acetate.

In another aspect, this document features a method for testing food for the presence of a food allergen of a human. The method comprises, or consists essentially of, (a) contacting a solid support comprising an immobilized anti-human IgE antibody with a food sample to be tested and a saliva sample from the human under conditions wherein the food allergen if present within the food sample binds to a human IgE antibody present in the saliva if the human is allergic to the food allergen and under conditions wherein a human IgE antibody if present within the saliva binds to the immobilized anti-human IgE antibody, thereby forming a complex comprising the anti-human IgE antibody, the human IgE antibody, and the food allergen if the human IgE antibody is present in the saliva and the food allergen is present in the food sample, (b) contacting the solid support with an anti-food allergen antibody comprising an enzyme under conditions wherein the anti-food allergen antibody binds to the complex if present, (c) removing unbound forms of the anti-food allergen antibody, (d) contacting the solid support with a substrate capable of being converted into a detectable label by the enzyme, and (e) detecting the detectable label, wherein the presence of the detectable label indicates that the food sample contains the food allergen. The food allergen can be a peanut, tree nut, walnut, almond, pecan, pistachio, hazelnut, Brazil nut, Macadamia nut, cashew, beechnut, chestnut, hickory nut, filberts, soy, milk, egg, wheat, or shellfish. The method can be performed in a restaurant setting. The anti-human IgE antibody can be a monoclonal antibody. The anti-food allergen antibody can be a polyclonal antibody. The step (b) can comprise contacting the solid support with multiple different anti-food allergen antibodies, wherein each of the multiple different anti-food allergen antibody comprises an enzyme and has a binding specificity for a different food allergen, and wherein each of the anti-food allergen antibody binds to the complex if the complex is present and if the complex comprises the food allergen for which the anti-food allergen antibody has binding specificity. The step (c) can comprise washing the solid support to remove the unbound forms of the anti-food allergen antibody. The solid support can be configured to have a reservoir housing a wash solution. The enzyme can be horseradish peroxidase.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a diagram of an example of a personal allergy detector device provided herein.

FIG. 2 is a diagram of the example of the personal allergy detector device of FIG. 1 within a protective cover.

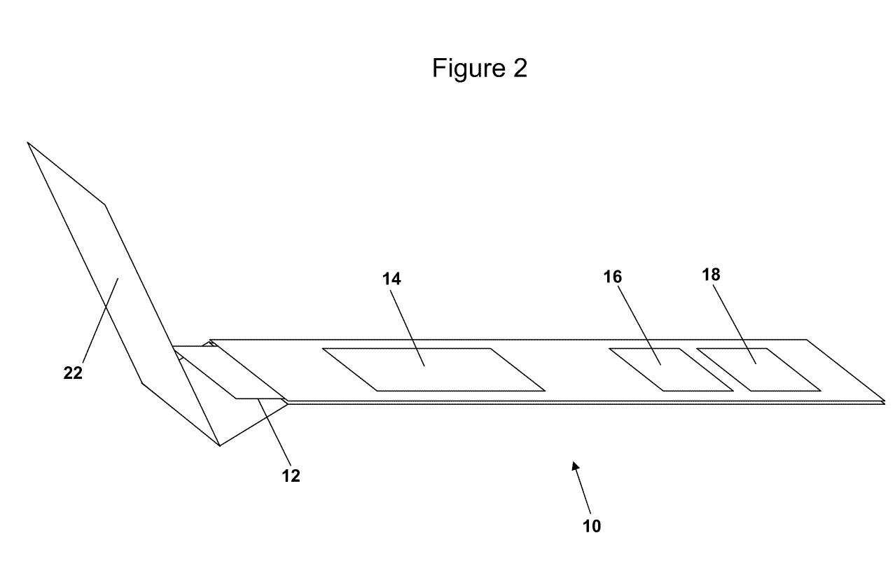

FIG. 3 is a diagram of the example of the personal allergy detector device of FIG. 1 that can result when a food allergen to be detected is present in the tested food sample.

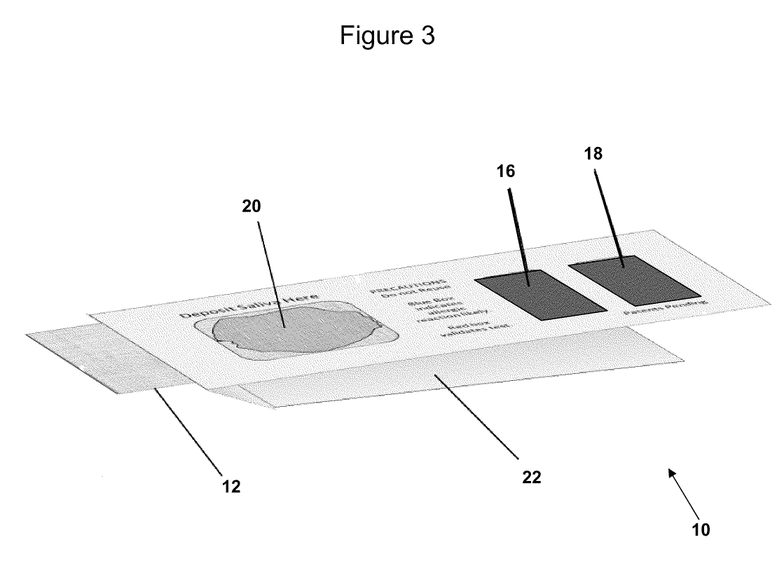

FIG. 4 is a diagram of the example of the personal allergy detector device of FIG. 1 that can result when a food allergen to be detected is not present in the tested food sample.

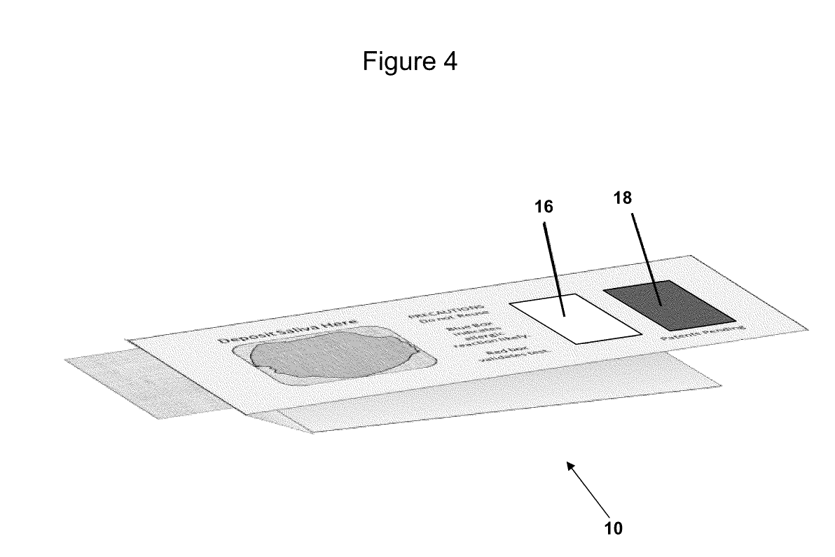

### DETAILED DESCRIPTION

This document relates to methods and materials involved in detecting allergens (e.g., food allergens). For example, this document provides methods and materials for assessing a food sample for the presence or absence of food allergens. Examples of food allergens that can be detected using the devices provided herein include, without limitation, peanuts, tree nuts (e.g., walnuts, almonds, pecans, pistachios, hazelnuts, Brazil nuts, Macadamia nuts, cashews, beechnuts, chestnuts, hickory nuts, and/or filberts), soy, milk, egg, wheat, and/or shellfish.

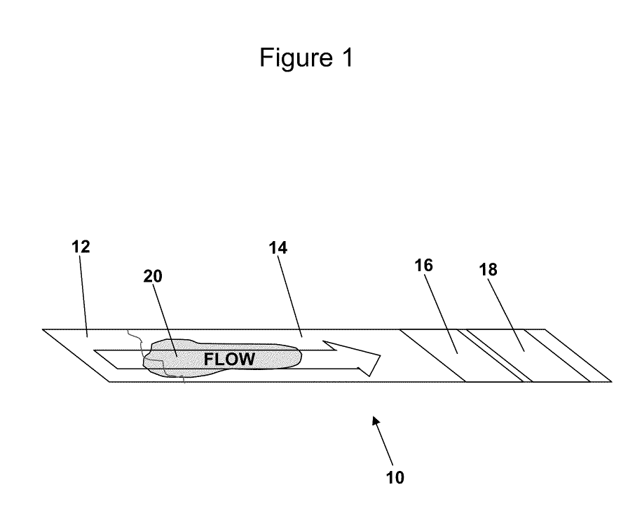

In one embodiment, a personal allergy detector device can be configured to detect a single allergen (e.g., an antigen to which a human is allergic), or a group of allergens simultaneously, in food(s), liquid(s), and/or pharmaceutical product(s). Such a device can include a solid support such as a strip of fibrous material (e.g., polyester clothe), a capillary membrane material, or a microfluidic device. In some cases, the solid support can include one or more membranes configured to allow molecules of different molecular weights to travel differently or to restrict a molecule of a particular molecular weight from advancing along a fluid path. With reference to FIG. 1, device 10 can include solid support 12 having a first zone 14, a second zone 16, and a third zone 18. The first zone 14 can include one or more different antibodies (e.g., two, three, four, five, six, or more antibodies) having the ability to bind a particular allergen (e.g., food allergen). Such anti-allergen antibodies can be monoclonal antibodies, polyclonal antibodies, or fragments thereof provided the fragment has the ability to bind a particular allergen. In this embodiment, the antibodies of the first zone can be present without being immobilized. For example, the antibodies of the first zone can be attached in a manner that allows them to be released into solution upon contact with a liquid sample to be tested. In addition, the anti-allergen antibodies of first zone 14 can be conjugated to an enzyme. Examples of such enzymes include, without limitation, horse radish peroxidase and alkaline phosphatase.

During use, a sample 20 to be tested (e.g., a food sample, a liquid sample, or a pharmaceutical sample) is applied in a fluid form to first zone 14 under conditions such that any allergens present in the sample bind to any antibodies present in first zone 14 that have the ability to bind to such an allergen. This binding can result in the formation of an allergen/anti-allergen antibody/enzyme complex. Given the fluid nature of the sample and the design of the solid support, the sample with any formed complexes and free anti-allergen antibody/enzyme conjugates can advance from first zone 14 to second zone 16.

The second zone 16 can include one or more different immobilized antibodies (e.g., two, three, four, five, six, or more antibodies) having the ability to bind a particular allergen (e.g., food allergen). Such immobilized anti-allergen antibodies can be monoclonal antibodies, polyclonal antibodies, or fragments thereof provided the fragment has the ability to bind a particular allergen. Second zone 16 can include immobilized substrate capable of forming a detectable label when in the presence of the enzyme conjugated to the anti-allergen antibodies of first zone 14. For example, when the anti-allergen antibodies of first zone 14 are conjugated to horseradish peroxidase, a substrate such as TMB (3,3′,5,5′-Tetramethylbenzidine), DAB (3,3′-Diaminobenzidine), or ABTS (2,2′-azino-bis(3-ethylbenzthiazoline-6-sulphonic acid)) can be attached to second zone 16.

During use, the presence of any allergen/anti-allergen antibody/enzyme complexes from first zone 14 that advances to second zone 16 can bind to the immobilized antibody that has binding specificity for the particular allergen of the allergen/anti-allergen antibody/enzyme complex, thereby immobilizing the complex to second zone 16. Immobilization of the complex that includes the enzyme to second zone 16 can allow for a detectable label to become visible to the user in second zone 16 (e.g., test zone). Thus, if an allergen to be detected is present in the sample being tested, then a detectable signal will be visible in second zone 16. Given the fluid nature of the sample and the design of the solid support, the sample with any formed and uncaptured complexes and free anti-allergen antibody/enzyme conjugates can advance further from second zone 16 to third zone 18.

The third zone 18 can include immobilized anti-antibody antibodies having the ability to bind the antibodies used in first zone 14 (e.g., goat, sheep, or recombinant human anti-mouse (or anti-recombinant human) Ig antibodies). Such immobilized anti-Ig antibodies can be monoclonal antibodies, polyclonal antibodies, or fragments thereof provided the fragment has the ability to bind the antibodies present in first zone 14. Third zone 18 can include an immobilized substrate capable of forming a detectable label when in the presence of the enzyme conjugated to the anti-allergen antibodies of first zone 14. For example, when the anti-allergen antibodies of first zone 14 are conjugated to horseradish peroxidase, a substrate such as TMB, DAB, or ABTS can be attached to third zone 18.

During use, the presence of any allergen/anti-allergen antibody/enzyme complexes from first zone 14 that advances to third zone 16 or any free anti-allergen antibody/enzyme conjugates that advance from first zone 14 that advances to third zone 16 can bind to the immobilized antibody that has binding specificity for the anti-allergen antibody, thereby immobilizing such antibodies conjugated to the enzyme to third zone 18. Such immobilization to third zone 18 can allow for a detectable label to become visible to the user in third zone 18 (e.g., control zone) and provide the user with confirmation that the device performed as intended. The presence of signal in second zone 16 and third zone 18 can indicate that an allergen is present, while the absence of signal in second zone 16 and the presence of a signal in third zone 18 can indicate that the allergens being tested for are absent. The absence of signal in third zone 18 can indicate that the test was incomplete and the user should consider repeating the test.

In some cases, a different color signal can be generated for each different allergen being tested. For example, an appropriate enzyme can be conjugated to an anti-peanut antigen antibody and an appropriate substrate for that enzyme can be used in second and third zones to create a red signal, and an appropriate enzyme can be conjugated to an anti-shellfish antigen antibody and an appropriate substrate for that enzyme can be used in second and third zones to create a blue signal.

In another embodiment, a personal allergy detector device can be configured to detect a single allergen, or a group of allergens simultaneously, in food(s), liquid(s), and/or pharmaceutical products based on the individual's antibody production. In this case, the personal allergy detector device can be individualized to a specific user and can be accomplished in several fashions. In one embodiment, an anti-IgE antibody (e.g., omalizumab) can be immobilized to a test strip. The user can place his or her salvia on the test strip, and the strip can be placed in a food, drink, or product to be tested. If IgE molecules are present in the user's saliva, these IgE molecules will bind to the immobilized anti-IgE antibodies. In addition, if an allergen recognized by the user's IgE molecules is present, then that IgE and antigen will bind to form a complex. As such, the allergen will be bound to the IgE molecule, and the IgE molecule will be bound to the immobilized anti-IgE antibody. A rinse is then performed to wash away additional unbound allergen and additional antibodies (e.g., IgG antibodies). A monoclonal or polyclonal anti-allergen antibody conjugated with a detectable label or enzyme is then added under conditions that allow the antibody to bind to the allergen is present. The test strip can be washed to remove any unbound anti-allergen antibodies. If the allergen is indeed bound to the antibody that is bound to the anti-IgE antibody, then the anti-allergen antibody with the detectable label can be visualized or the enzyme can be used to generate a detectable signal. In some cases, a different color signal can be generated for each different allergen being tested. In addition, multiple anti-allergen antibodies could be added depending on the food allergies that a patient has. Examples of detectable labels include, without limitation, propidium iodide (PI), fluorescein, fluorescein isothiocyanate (FITC), tetramethylrhodamine isothiocyanate (TRITC), rhodamine, R-phycoerythrin, and aminomethylcoumarin acetate (AMCA).

In another embodiment, a personal allergy detector device can be configured to have anti-allergen antibodies immobilize to a solid support (e.g., a test strip). Saliva and a sample to be tested can then be added to the solid support. At that point, if the allergen is present in the sample being tested, it would bind to the immobilized anti-allergen antibody. Such an antibody can be a monoclonal antibody, a polyclonal antibody, or a fragment thereof. Subsequently, if IgE molecules were present in a user's saliva, then they would bind the allergen that is bound to the anti-allergen antibody if that user is allergic to that allergen. A rinse can be performed to remove any unbound material (e.g., IgG antibodies). An anti-human IgE antibody conjugated with a detectable label or enzyme can be added. This antibody would attach to the human IgE antibodies that are bound to allergen that are bound to the anti-allergen antibodies. A rinse can be performed to remove any unbound material. The remaining anti-human IgE antibodies with the detectable label can be visualized or the enzyme can be used to generate a detectable signal.

The devices provided herein can be used to detect simultaneously multiple allergens that a user may be suffering from. In addition, the devices provided herein can be used at the point of interest (e.g., a restaurant) without requiring any medically trained professionals. In some cases, the device can be individualized as described herein to the patient using his or her saliva.

The invention will be further described in the following examples, which do not limit the scope of the invention described in the claims.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for testing food for the presence of a food allergen specific to a human, wherein said method comprises:
(a) contacting a solid support comprising an immobilized anti-human IgE antibody with a food sample to be tested and a saliva sample from said human under conditions wherein said food allergen if present within said food sample binds to a human IgE antibody present in said saliva if said human is allergic to said food allergen and under conditions wherein a human IgE antibody if present within said saliva binds to said immobilized anti-human IgE antibody, thereby forming a complex comprising said anti-human IgE antibody, said human IgE antibody, and said food allergen if said human IgE antibody is present in said saliva and said food allergen is present in said food sample,
(b) contacting said solid support with an anti-food allergen antibody comprising a detectable label under conditions wherein said anti-food allergen antibody binds to said complex if present,
(c) removing unbound forms of said anti-food allergen antibody, and
(d) detecting said detectable label, wherein the presence of said detectable label indicates that said food sample contains said food allergen.

2. The method of claim 1, wherein said food allergen is a peanut, tree nut, walnut, almond, pecan, pistachio, hazelnut, Brazil nut, Macadamia nut, cashew, beechnut, chestnut, hickory nut, filberts, soy, milk, egg, wheat, or shellfish.

3. The method of claim 1, wherein said method is performed in a restaurant setting.

4. The method of claim 1, wherein said anti-human IgE antibody is a monoclonal antibody.

5. The method of claim 1, wherein said anti-food allergen antibody is a polyclonal antibody.

6. The method of claim 1, wherein said step (b) comprises contacting said solid support with multiple different anti-food allergen antibodies, wherein each of said multiple different anti-food allergen antibody comprises a detectable label and has a binding specificity for a different food allergen, and wherein each of said anti-food allergen antibody binds to said complex if said complex is present and if said complex comprises the food allergen for which said anti-food allergen antibody has binding specificity.

7. The method of claim 1, wherein said step (c) comprises washing said solid support to remove said unbound forms of said anti-food allergen antibody.

8. The method of claim 1, wherein said solid support is configured to have a reservoir housing a wash solution.

9. The method of claim 1, wherein said detectable label is propidium iodide, fluorescein, fluorescein isothiocyanate, tetramethylrhodamine isothiocyanate, rhodamine, R-phycoerythrin, or aminomethylcoumarin acetate.

10. A method for testing food for the presence of a food allergen of a human, wherein said method comprises:
(a) contacting a solid support comprising an immobilized anti-human IgE antibody with a food sample to be tested and a saliva sample from said human under conditions wherein said food allergen if present within said food sample binds to a human IgE antibody present in said saliva if said human is allergic to said food allergen and under conditions wherein a human IgE antibody if present within said saliva binds to said immobilized anti-human IgE antibody, thereby forming a complex comprising said anti-human IgE antibody, said human IgE antibody, and said food allergen if said human IgE antibody is present in said saliva and said food allergen is present in said food sample,
(b) contacting said solid support with an anti-food allergen antibody comprising an enzyme under conditions wherein said anti-food allergen antibody binds to said complex if present,
(c) removing unbound forms of said anti-food allergen antibody,
(d) contacting said solid support with a substrate capable of being converted into a detectable label by said enzyme, and
(e) detecting said detectable label, wherein the presence of said detectable label indicates that said food sample contains said food allergen.

11. The method of claim 10, wherein said food allergen is a peanut, tree nut, walnut, almond, pecan, pistachio, hazelnut, Brazil nut, Macadamia nut, cashew, beechnut, chestnut, hickory nut, filberts, soy, milk, egg, wheat, or shellfish.

12. The method of claim 10, wherein said method is performed in a restaurant setting.

13. The method of claim 10, wherein said anti-human IgE antibody is a monoclonal antibody.

14. The method of claim 10, wherein said anti-food allergen antibody is a polyclonal antibody.

15. The method of claim 10, wherein said step (b) comprises contacting said solid support with multiple different anti-food allergen antibodies, wherein each of said multiple different anti-food allergen antibody comprises an enzyme and has a binding specificity for a different food allergen, and wherein each of said anti-food allergen antibody binds to said complex if said complex is present and if said complex comprises the food allergen for which said anti-food allergen antibody has binding specificity.

16. The method of claim 10, wherein said step (c) comprises washing said solid support to remove said unbound forms of said anti-food allergen antibody.

17. The method of claim 10, wherein said solid support is configured to have a reservoir housing a wash solution.

18. The method of claim 10, wherein said enzyme is horseradish peroxidase.
